(19) United States
(12) Patent Application Publication
(10) Pub. No.: US 2018/0350456 A1
(43) Pub. Date: Dec. 6, 2018

(54) PATIENT CENTERED DATA HOME

(71) Applicant: Strategic Health Information Exchange Collaborative, Grand Junction, CO (US)

(72) Inventors: David Kendrick, Tulsa, OK (US); Melissa Kotrys, Phoenix, AZ (US); Dick Thompson, Grand Junction, CO (US); Teresa Rivera, Salt Lake City, UT (US)

(21) Appl. No.: 15/994,841

(22) Filed: May 31, 2018

Related U.S. Application Data
(60) Provisional application No. 62/513,314, filed on May 31, 2017.

(51) Int. Cl. G16H 10/60

(52) U.S. Cl. CPC G16H 10/60

(57) ABSTRACT

The invention provides systems and methods that quickly and reliably convey more complete, longitudinal healthcare information relating to a patient stored in the patient's home health information exchange (HIE) to users of a different HIE and vice versa. The systems and methods of the invention are compatible with a wide range of existing hardware and software solutions for the storage and dissemination of healthcare information.

### CROSS-REFERENCE TO RELATED APPLICATION

This application claims the benefit of U.S. Provisional Patent Application 62/513,314, filed 31 May 2017, the entirety of which is incorporated herein by reference.

### FIELD OF THE INVENTION

This invention relates to systems and methods for providing longitudinal healthcare information relating to an identified patient, and particularly to systems and methods for providing health care information relating to a patient stored in the patient's home health information exchange (HIE) to users of a different HIE and vice versa.

### BACKGROUND OF THE INVENTION

In the United States, a health information exchange (HIE) is an organization that facilitates the exchange of health care information between hospitals, healthcare providers, and healthcare and human service organizations within a defined geographic region; the geographic region is typically a state, or a part of a state. Typically, an HIE shares information among individuals and organizations within its geographic region according to predetermined policies and procedures, in addition to local, state, and federal laws and regulations pertaining to, e.g., consent and permitted use.

Because each HIE and geographic community sets its own policies and procedures for the sharing of healthcare information and maintains its own separate hardware and software for the storage and dissemination of that information, transfer of data between different HIEs, or between individuals and/or organizations served by different HIEs, is frequently difficult or impossible. This difficulty is especially apparent when a patient seeks medical care from a hospital or healthcare provider outside the geographic region served by the patient's “home” HIE, i.e. the HIE that is primarily responsible for storing the patient's healthcare information. Such “border crossings” are very common and may occur in any number of circumstances, including but not limited to circumstances in which the patient becomes injured or ill while traveling, seeks a specialty or quality of care not available in the region served by his or her home HIE, or maintains two or more homes in different areas of the country. In such circumstances, the consequences of any delay, interruption, or disconnection in the transfer of healthcare information from one HIE to another can range from merely inconvenient to life-threatening.

There is thus a need in the art for systems and methods that quickly and reliably convey more complete, longitudinal healthcare information relating to a patient stored in the patient's home HIE to users of a different HIE and vice versa. It is further advantageous for such systems and methods to be compatible with a wide range of existing hardware and software solutions for the storage and dissemination of healthcare information.

### SUMMARY OF THE INVENTION

The present invention provides systems and methods for quickly and reliably providing more complete, longitudinal healthcare information relating to a patient, and particularly to systems and methods for providing healthcare information relating to an identified patient stored in a patient's home health information exchange (HIE) to users of a different (“away”) HIE and vice versa. The systems and methods of the present invention may automatically supply specific, essential triggers regarding a patient's healthcare information to specific users of one or more

HIEs, based on such information as, by way of non-limiting example, whether a clinical intervention relating to the patient is occurring or has occurred, where and when the intervention and/or a related event occurred, and the nature of the intervention and/or related event. Systems and methods of the present invention may further allow a user of one or more of HIEs to “target” follow-up information requests and updates of a patient's health record, and for such queries and updates to be accessible across multiple HIEs and other healthcare information and/or record locator systems.

Systems and methods of the present invention may be implemented across existing HIEs and utilize conventional technical solutions and infrastructures previously known to those skilled in the art. Further, the systems and methods of the present invention may provide additional features, including but not limited to real-time event surveillance, exception/anomaly processing, and event notification, and matching of personal records across multiple disparate master identification systems without the use of a single “global” identifier.

The general Patient Centered Data Home (PCDH) methodology is based on routing of patient healthcare information according to the patient's home ZIP code. It enables any HIE that is part of a PCDH system to connect with the other HIEs of the PCDH system, regardless of the electronic health record (EHR) and HIE platforms used by the individual HIEs. This methodology creates a record locator service for future requests and delivery of critical clinical information, creating a more complete patient longitudinal record.

It is one aspect of the present invention to provide a PCDH system, comprising a home HIE, comprising healthcare information of a patient; and an away HIE, configured to receive patient healthcare information from one or more authorized data sources of the away HIE, wherein the away HIE, upon receiving a triggering message pertaining to a patient that resides outside of its geographic region and according to a first algorithm, identifies the patient to whom the triggering message pertains and transmits a request for healthcare information to the home HIE, wherein the home HIE, upon receiving the request and according to a second algorithm, fetches the requested healthcare information and transmits the requested healthcare information to the away HIE, wherein the away HIE, upon receiving the requested healthcare information, transmits the requested healthcare information to the one or more of its authorized users, and wherein both away and home HIEs follow their community's policies and procedures in respect of sharing clinical information of the patient.

In embodiments, upon receiving the request, the home HIE may determine, according to a third algorithm, which of one or more authorized users of the home HIE should receive alerts or additional appropriate healthcare information relating to the patient and transmits an alert or other additional healthcare information to the determined authorized users of the home HIE.

In embodiments, the away HIE, upon receiving updated healthcare information of the patient from its data sources, may transmit the updated healthcare information of the patient to the home HIE.

In embodiments, the triggering message may comprise at least one of hospital admission information, episode-of-care information, discharge information, and post-discharge results information.

In embodiments, the home HIE may transmit follow-up information to the away HIE.

In embodiments, the triggering message received by the away HIE may be sent by a healthcare entity within the away HIE's geographic region. The triggering message may comprise a request by the healthcare entity for at least a portion of a medical record of the patient.

In embodiments, at least one of the one or more authorized users of the home HIE may be a healthcare entity within the home HIE's geographic region. The alert or other additional healthcare information may comprise an update to at least a portion of a medical record of the patient.

In embodiments, the home HIE and away HIE may use at least one of different electronic health record platforms and different HIE platforms.

It is another aspect of the present invention to provide a method of sharing healthcare information of a patient, comprising receiving, by an away HIE from one or more authorized data sources of the away HIE, a triggering message pertaining to a patient; identifying the patient, according to a first algorithm; creating a record locator service for future query; transmitting, by the away HIE, a request for healthcare information pertaining to a patient to a home HIE; fetching the requested healthcare information, according to a second algorithm; transmitting, by the home HIE, the requested healthcare information to the away HIE; and transmitting, by the away HIE, the requested healthcare information to the one or more authorized users of the away HIE.

In embodiments, the method may further comprise determining, by the home HIE, which of one or more authorized users of the home HIE should receive alerts or additional healthcare information relating to the patient, according to a third algorithm; and transmitting, by the home HIE, an alert or additional healthcare information to the determined authorized users of the home HIE.

In embodiments, the method may further comprise receiving, by the away HIE from the one or more authorized users of the away HIE, updated healthcare information of the patient; and transmitting, by the away HIE, the updated healthcare information of the patient to the home HIE.

In embodiments, the triggering message may comprise at least one of hospital admission information, episode-of-care information, discharge information, and post-discharge results information.

In embodiments, the method may further comprise transmitting, by the home HIE, follow-up information to the away HIE.

In embodiments, the triggering message received by the away HIE may be sent by a healthcare entity within the away HIE's geographic region. The triggering message may comprise a request by the healthcare entity for at least a portion of a medical record of the patient.

In embodiments, at least one of the one or more authorized users of the home HIE may be a healthcare entity within the home HIE's geographic region. The alert or other additional healthcare information may comprise an update to at least a portion of a medical record of the patient.

In embodiments, the home HIE and away HIE may use at least one of different electronic health record platforms and different HIE platforms.

### BRIEF DESCRIPTION OF THE DRAWINGS

FIG. 1 illustrates a Patient Centered Data Home™ (PCDH) system, and a method of using the PCDH system to share health care information across multiple HIEs, according to embodiments of the present invention.

### DETAILED DESCRIPTION OF THE INVENTION

As used herein, the terms “Patient Centered Data Home™” and “PCDH” are interchangeable and each refer to any system or method that enables rapid and reliable communication and access of healthcare information between two or more health information exchanges (HIEs).

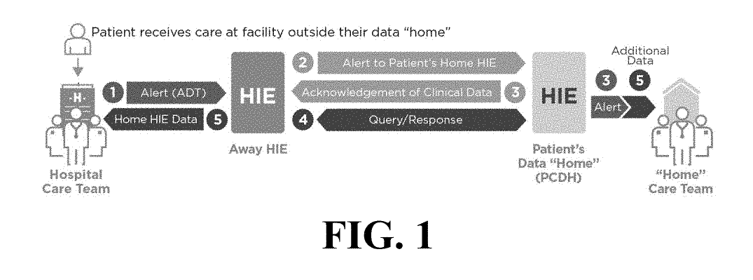

Referring now to FIG. 1, a PCDH system and methods of use thereof are illustrated. In the embodiment illustrated in FIG. 1, the PCDH is used to transmit health information upon a patient's receiving care from a healthcare provider, in this case a hospital, outside the geographic region served by his or her “home” HIE. In step 1, the hospital's electronic health record (EHR) system automatically sends a first alert, comprising information related to the patient, to the HIE that covers the geographic region where the hospital is located, labeled as the “away” HIE; in the exemplary embodiment as described, the information related to the patient comprises an admission, discharge, and treatment (ADT) record, but any appropriate type of healthcare information may be sent, including, by way of non-limiting example, hospital admission information, episode-of-care information, discharge information, and post-discharge results information. The away HIE, according to an algorithm based on zip code mapping, identifies the patient to whom the ADT pertains and determines, based on one or more pieces of information contained in the ADT record and/or information previously stored by the away HIE, that the patient's home ZIP code is located outside the geographic region served by the away HIE. In step 2, the away HIE then transmits a second alert comprising the information contained in the ADT to the patient's home HIE; where the PCDH comprises three or more HIEs, step 2 further comprises a substep in which the PCDH system determines, according to an algorithm, which HIE is the patient's home HIE and transmits the information to that HIE alone. In step 3, the home HIE transmits to the away HIE an acknowledgment and/or confirmation that the second alert has been received. The home HIE then determines, according to an algorithm, which of its users should be notified of the receipt of new information pertaining to the patient, e.g. the patient's primary care physician, and, in the same step 3, transmits a third alert and/or the appropriate healthcare information to those users. The home HIE also, according to the home HIE's policies and procedures (which may include patient consent to share clinical information), fetches information pertaining to the patient and necessary for treatment stored in the home HIE and transmits that information to the away HIE in step 4, which subsequently transmits the information to the authorized representative in step 5. During or after the patient's care in the hospital, the away HIE sends further information, such as discharge information, to the home HIE to update the patient's medical record in the home HIE; the home HIE may then determine, according to an algorithm, whether to transmit a further alert and/or appropriate healthcare information to the previously identified users of the HIE, in an optional substep of step 5.

Upon completion of the indicated steps, both the home and away HIEs, as well as other HIEs (if any) that are part of the same PCDH system, may create and store a link between patient IDs, which may indicate to each of the HIEs, for example, that a patient identified by a first ID number by the home HIE and by at least a second ID number by one or more away HIEs are the same patient. Such a link between identifiers may enable faster and more thorough completion of steps 1-6 when the patient seeks care in the geographic region represented by the same or a different HIE in the future, as well as sharing of additional data relating to the patient.

The system and method illustrated in FIG. 1 and described in the preceding paragraphs are exemplary and non-exhaustive, and those of ordinary skill in the art will understand other schemas for routing and sharing information that are within the scope of the present invention. The general PCDH methodology based on routing of patient healthcare information according to the patient's home ZIP code enables any HIE that is part of a PCDH system to connect with the other HIEs of the PCDH system, regardless of the electronic health record (EHR) and HIE platforms used by the individual HIEs, while respecting regional policies such as patient consent developed by the local communities.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described above is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A Patient Centered Data Home (PCDH) system, comprising:
a home HIE, comprising healthcare information of a patient; and
an away HIE, configured to receive patient healthcare information from one or more authorized data sources of the away HIE,
wherein the away HIE, upon receiving a triggering message pertaining to a patient that resides outside of its geographic region and according to a first algorithm, identifies the patient to whom the triggering message pertains and transmits a request for healthcare information to the home HIE,
wherein the home HIE, upon receiving the request and according to a second algorithm, fetches the requested healthcare information and transmits the requested healthcare information to the away HIE,
wherein the away HIE, upon receiving the requested healthcare information, transmits the requested healthcare information to the one or more of its authorized users, and
wherein both away and home HIEs follow their community's policies and procedures in respect of sharing clinical information of the patient.

2. The PCDH system of claim 1, wherein, upon receiving the request, the home HIE determines, according to a third algorithm, which of one or more authorized users of the home HIE should receive alerts or additional appropriate healthcare information relating to the patient and transmits an alert or other additional healthcare information to the determined authorized users of the home HIE.

3. The PCDH system of claim 1, wherein the away HIE, upon receiving updated healthcare information of the patient from its data sources, transmits the updated healthcare information of the patient to the home HIE.

4. The PCDH system of claim 1, wherein the triggering message comprises at least one of hospital admission information, episode-of-care information, discharge information, and post-discharge results information.

5. The PCDH system of claim 1, wherein the home HIE transmits follow-up information to the away HIE.

6. The PCDH system of claim 1, wherein the triggering message received by the away HIE is sent by a healthcare entity within the away HIE's geographic region.

7. The PCDH system of claim 6, wherein the triggering message comprises a request by the healthcare entity for at least a portion of a medical record of the patient.

8. The PCDH system of claim 2, wherein at least one of the one or more authorized users is a healthcare entity within the home HIE's geographic region.

9. The PCDH system of claim 8, wherein the alert or other additional healthcare information comprises an update to at least a portion of a medical record of the patient.

10. The PCDH system of claim 1, wherein the home HIE and away HIE use at least one of different electronic health record platforms and different HIE platforms.

11. A method of sharing healthcare information of a patient, comprising:
receiving, by an away HIE from one or more authorized data sources of the away HIE, a triggering message pertaining to a patient;
identifying the patient, according to a first algorithm;
creating a record locator service for future query;
transmitting, by the away HIE, a request for healthcare information pertaining to a patient to a home HIE;
fetching the requested healthcare information, according to a second algorithm;
transmitting, by the home HIE, the requested healthcare information to the away HIE; and
transmitting, by the away HIE, the requested healthcare information to the one or more authorized users of the away HIE.

12. The method of claim 11, further comprising:
determining, by the home HIE, which of one or more authorized users of the home HIE should receive alerts or additional healthcare information relating to the patient, according to a third algorithm; and
transmitting, by the home HIE, an alert or additional healthcare information to the determined authorized users of the home HIE.

13. The method of claim 11, further comprising:
receiving, by the away HIE from the one or more authorized users of the away HIE, updated healthcare information of the patient; and
transmitting, by the away HIE, the updated healthcare information of the patient to the home HIE.

14. The method of claim 11, wherein the triggering message comprises at least one of hospital admission information, episode-of-care information, discharge information, and post-discharge results information.

15. The method of claim 11, further comprising transmitting, by the home HIE, follow-up information to the away HIE.

16. The method of claim 11, wherein the triggering message received by the away HIE is sent by a healthcare entity within the away HIE's geographic region.

17. The method of claim 16, wherein the triggering message comprises a request by the healthcare entity for at least a portion of a medical record of the patient.

18. The method of claim 12, wherein at least one of the one or more authorized users is a healthcare entity within the home HIE's geographic region.

19. The method of claim 18, wherein the alert or other additional healthcare information comprises an update to at least a portion of a medical record of the patient.

20. The method of claim 11, wherein the home HIE and away HIE use at least one of different electronic health record platforms and different HIE platforms.
